(19) United States
(12) Patent Application Publication
(10) Pub. No.: US 2008/0139531 A1
(43) Pub. Date: Jun. 12, 2008

(54) USE OF CONNECTIVE TISSUE MAST CELL STABILIZERS TO FACILITATE OCULAR SURFACE RE-EPITHELIZATION AND WOUND REPAIR

(75) Inventors: John M. Yanni, Burleson, TX (US); Daniel A. Gamache, Arlington, TX (US); Steven T. Miller, Arlington, TX (US)

Correspondence Address: ALCON, IP LEGAL, TB4-8, 6201 SOUTH FREEWAY, FORT WORTH, TX, 76134, US

(73) Assignee: ALCON MANUFACTURING LTD., Fort Worth, TX (US)

(21) Appl. No.: 11/947,041

(22) Filed: Nov. 29, 2007

Related U.S. Application Data
(60) Provisional application No. 60/872,715, filed on Dec. 4, 2006.

(51) Int. Cl. A61K 31/55; A61K 31/335; A61K 31/519; A61K 31/505; A61K 31/4422

(52) U.S. Cl. 514/214.02; 514/450; 514/258.1; 514/256; 514/356

(57) ABSTRACT

Disclosed are methods of treating a wound in a subject that involve administering to the subject a pharmaceutically effective amount of a composition that includes one or more human connective tissue mast cell stabilizers, wherein administration of the composition results in treatment of the wound. In particular embodiments, the wound is an ophthalmic or dermal wound, such as a corneal epithelial defect, a conjunctival wound, or dermal abrasion. Administration, for example, may be by topical application of the composition to the ocular surface or skin. Exemplary mast cell stabilizers include olopatadine, variants of olopatadine, alcaftidine, derivatives of alcaftidine, dihydropyridines, and spleen tyrosine kinase inhibitors.

This application claims priority to U.S. Provisional Application, U.S. Ser. No. 60/872,715 filed Dec. 4, 2006.

### BACKGROUND OF THE INVENTION

1. Field of the Invention

The present invention relates generally to the field of mast cell stabilizers, pharmaceutics, and the treatment and prevention of wounds. More particularly, the present invention concerns methods of treating or preventing a wound in a subject that involve administering a pharmaceutically effective amount of a composition comprising one or more mast cell stabilizers to the wound of the subject.

2. Description of Related Art

Wounds of the ocular surface, such as a corneal epithelial defect, are exceedingly common and occur under many different circumstances. For example, a corneal epithelial defect may be due to trauma, infection, a side effect of allergic conjunctivitis, contact lens wear, exposure keratopathy, or ocular surgery. Measures to promote healing are numerous, and include the discontinuation of potentially toxic medications and the use of ophthalmic lubricants, anti-infective agents, and anti-inflammatory agents. Other measures to promote healing include the protection of the ocular surface, such as through the use of a patch, a bandage contact lens and tarsorrhaphy.

Although many patients respond to conventional therapies, there remains a significant subset of patients who fail conventional therapies. Failure of the wound to heal can be extremely frustrating for both patient and physician.

New therapies are under investigation, and include nerve growth factors, the use of fetal cord blood, the use of amniotic membrane, and the use of limbal stem cell grafting (reviewed in www.uveitis.org/medical/articles/clinical/growth.html). It is not yet known whether any of these measures will result in effective treatment of an ocular surface wound. Thus, there is the need for more effective treatments of ocular surface wounds.

Mast cells are a type of cell that is known to be involved in inflammatory responses. A wide variety of stimuli may cause the activation of mast cells, and subsequently cause them to migrate to a particular location and/or to undergo de-granulation. These stimuli may be immunologic (such as antibodies or allergens) or non-immunologic (such as chemical agents) in nature.

The precise role of mast cells in wound healing is unknown. Understanding the role of mast cells, if any, in wound healing is made difficult by the fact that mast cell types differ within species. Further, mast cell populations exist within the same species which differ in phenotype, biochemical properties, functional responses, and pharmacological responses. For example, one subtype of mast cells, MC_{TC}, is known to be present in the skin and conjunctiva, but not in other tissues.

Chymase, a serine protease found in MC_{TC} mast cells, is an important marker for human mast cells as well as a mediator of inflammation and matrix remodelling (Buckley et al., 1999). It has been suggested that skin mast cells and mast cell chymase participates in the healing process as well as in fibrotic skin disease (Nishikori et al., 1998). In particular, it has been reported that the healing process in an animal model of wound healing corresponded strongly with mast cell density and chymase activity in both acute and subacute phases of wound healing. Recently, however, it has been shown that chymase released from activated mast cells may inhibit the repair of scratch-damaged monolayers of epithelial cells in vitro (Gordon, 2005). Thus, the precise role of mast cell chymase in corneal epithelial wound healing is unknown.

### SUMMARY OF THE INVENTION

The present invention overcomes drawbacks of the prior art by providing for novel methods of treating wounds in a subject, such as ocular surface wounds and skin wounds. In particular, the inventors have found that inhibitors of mast cell chymase release can be applied in the treatment of wounds in a subject. For example, topical olopatadine, a mast cell stabilizer that inhibits mast cell chymase release, can aid in the process of corneal and/or conjunctival re-epithelialization and wound repair.

The invention set forth herein is generally directed to a method of treating a wound in a subject, involving administering to the subject a pharmaceutically effective amount of a composition that includes one or more mast cell stabilizers, wherein administration of the pharmaceutically effective amount of the composition results in treatment of the wound. In a particular embodiment, the wound is an ophthalmic wound. In another embodiment, the wound is a skin wound.

A “subject” refers to either a human or non-human, such as primates, mammals, and vertebrates. In particular embodiments, the subject is a human.

A “wound” is defined herein to refer to a break or disruption in the continuity of cells of a tissue surface of a subject. Any cause of disruption of the continuity of the cells of a tissue surface of a subject is contemplated by this definition. For example, the disruption may be caused by surgery or trauma. Other causes of wounds include infections and inflammation. The surface of the organ may be the surface of the eye, such as the corneal surface or the conjunctival surface.

Examples of cells that make up the tissue surface include epithelial cells and mucosal cells. The disruption of the cells that may up the tissue surface may either involve the full-thickness of the cell layer or may be involve only a portion of the cell layer. For example, included in the definition of “wound” is a corneal epithelial defect that is full-thickness (i.e., a defect to the basement membrane of the corneal), or a partial disruption of the corneal epithelial layer, and includes corneal abrasions and corneal ulcers.

In a particular embodiment, the wound is a corneal wound. For example, the corneal wound may further be defined as a corneal epithelial defect, a recurrent corneal erosion, or a corneal ulcer. Included within the definition of corneal epithelial defect is punctate epithelial keratopathy. The corneal epithelial defect may be due to any cause or be associated secondarily with any ophthalmic condition. For example, the corneal epithelial defect may be caused by trauma (surgical or nonsurgical), infection (e.g., bacterial, viral, or fungal keratitis), inflammation (e.g., secondary to allergic conjunctivitis, giant papillary conjunctivitis, vernal conjunctivitis, atopic keratoconjunctivitis), chemical or ultraviolet exposure of the cornea, or dry eye syndrome. The defect may also be associated with the use of preserved topical products, keratitis, blepharitis, uveitis, tear film instability, or be iatrogenic. In further particular embodiments, the corneal epithelial defect is a persistent corneal epithelial defect that has been unresponsive to conventional therapy.

In another embodiment, the wound is a conjunctival wound. For example, the conjunctival wound may be a wound associated with ophthalmic surgery, trauma, or conjunctival disease such as allergic conjunctivitis, giant papillary conjunctivitis, vernal conjunctivitis, or atopic keratoconjunctivitis.

In further embodiments, the wound is a skin wound. The skin wound may be the result of trauma (surgical or nonsurgical), infection, inflammation, burn, or primary skin disease. Examples of primary skin diseases include blistering diseases such as epidermolysis bullosa and bullous pemphigoid.

Treating a wound includes restoration of the integrity of the cells of the tissue surface of the subject. For example, treatment of an epithelial defect involves restoration of the continuity of the corneal epithelium.

A “mast cell stabilizer” is defined herein to refer to an agent that inhibits the degranulation of sensitized and/or nonsensitized mast cells. A mast cell stabilizer thus inhibits the release of inflammatory mediators, such as histamine, SRS-A, and chymase from mast cells. A wide variety of mast cell stabilizers are known in the art. These agents are known in the art as antiasthmatic and antiallergic agents. However, only mast cell stabilizers effective in human tryptase- and chymase-containing mast cells (connective tissue type) are effective in the methods of the present invention. One of ordinary skill in the art would be familiar with this class of agents.

Exemplary mast cell stabilizers include olopatadine, derivatives of olopatadine, alcaftidine, derivatives of alcaftadine, spleen tyrosine kinase inhibitors, and dihydropyridines. In particular embodiments, the mast cell stabilizer is olopatadine.

In further embodiments, the mast cell stabilizer is a derivative of olopatadine. For example, the derivative of olopatadine may be (Z)-11-(3-(dimethylamino)propylidene)-6,11-dihydrodibenz[b,e]oxepin-2-carboxylic acid, (E)-11-(3 (dimethylamino)propylidene)-6,11-dihydrodibenz[b,e]oxepin-2-carboxylic acid, (E)-11-(3-(dimethylamino)propylidene)-6,11-dihydrodibenz[b,e]oxepin-3-carboxylic acid, (Z)-11-(3-(dimethylamino)propylidene)-6,11-dihydrodibenz[b,e]oxepin-3-carboxylic acid, (E)-11-(3-(dimethylamino)propylidene)-6,11-dihydrodibenz[b,e]oxepin-8-carboxylic acid, (Z)-11-(3-(dimethylamino)propylidene)-6,11-dihydrodibenz[b,e]oxepin-8-carboxylic acid, (E)-11-(3-(dimethylamino)propylidene)-6,11-dihydrodibenz[b,e]oxepin-9-carboxylic acid, (Z)-11-(3-(dimethylamino)propylidene)-6,11-dihydrodibenz[b,e]oxepin-9-carboxylic acid, (E)-11-(3-(dimethylamino)propylidene)-6,11-dihydrodibenz[b,e]oxepin-2-acrylic acid, (Z)-11-(3-(dimethylamino)propylidene)-6,1-dihydrodibenz[b,e]oxepin-2-acrylic acid, (E)-5-(3-(dimethylamino)propylidene)-10,11-dihydro-5H-dibenzo[a,d]cyclohepten-3-carboxylic acid, and (Z)-5-(3-(dimethylamino)propylidene)-10,11-dihydro-5H-dibenzo[a,d]cyclohepten-3-carboxylic acid.

In particular embodiments, the derivative of doxepin is 11-(3-dimethylaminopropylidene)-6,11-dihydrodibenz[b,e]oxepine-2-carboxylic acid, 11-(3-dimethylaminopropylidene)-6,11-dihydrodibenz[b,e]oxepine-2(E)-acrylic acid, 5,6-dihydro-11-(1-methyl-4-piperidinylidene)-11H-imidazo[2,1-b][3]benzazepine, 9-fluoro-6,11-dihydro-11-(1-methyl-4-piperidinylidene)-5H-imidazo[2,1-b][3]benzazepine, 11-(1-methyl-4-piperidinylidene)-11H-imidazo[2,1-b][3]benzazepine, 6,11-dihydro-11-(1-methyl-4-piperidinylidene)-5H-imidazo[2,1-b][3]benzazepine-3-methanol, 8-fluoro-6,11-dihydro-11-(1-methyl-4-piperidinylidene)-5H-imidazo[2,1-b][3]benzazepine, 6,11-dihydro-11-(1-methyl-4-piperidinylidene)-5H-imidazo[2,1-b][3]benzazepine-3-carboxaldehyde, 6,11-dihydro-11-(1-methyl-4-piperidinylidene)-5H-imidazo[2,1-b][3]benzazepine-3 carboxylic acid, 7-fluoro-6,11-dihydro-11-(1-methyl-4-piperidinylidene)-5H-imidazo[2,1-b][3]benzazepine, 4-(8-fluoro-5,6-dihydro-11H-imidazo[2,1-b][3]benzazepin-11-ylidene)-1-piperidinepropanoic acid dihydrate, methyl cis-11-(3-dimethylaminopropylidene)-6,11-dihydrodibenz[b,e]oxepin-2-carboxylate, methyl trans-11-(3-dimethylaminopropylidene)-6,11-dihydrodibenz[b,e]oxepin-2-carboxylate, ethyl cis-11-(3-dimethylaminopropylidene)-6,11-dihydrodibenz[b,e]oxepin-2-carboxylate, ethyl trans-11-(3-dimethylaminopropylidene)-6,11-dihydrodibenz[b,e]oxepin-2-carboxylate, cis-11-(3-dimethylaminopropylidene)-6,11-dihydrodibenz[b,e]oxepin-2-carboxylic acid, trans-11-(3-dimethylaminopropylidene)-6,11-dihydrodibenz[b,e]oxepin-2-carboxylic acid, methyl cis-11-(3-diethylaminopropylidene)-6,11-dihydrodibenz[b,e]oxepin-2-carboxylate, methyl trans-11-(3-diethylaminopropylidene)-6,11-dihydrodibenz[b,e]oxepin-2-carboxylate, cis-11-(3-diethylaminopropylidene)-6,11-dihydrodibenz[b,e]oxepin-2-carboxylic acid, trans-11-(3-diethylaminopropylidene)-6,11-dihydrodibenz[b,e]oxepin-2-carboxylic acid, methyl cis-11-(3-pyrrolidinopropylidene)-6,11-dihydrodibenz[b,e]oxepin-2-carboxylate, methyl trans-11-(3-pyrrolidinopropylidene)-6,11-dihydrodibenz[b,e]oxepin-2-carboxylate, cis-11-(3-pyrrolidinopropylidene)-6,11-dihydrodibenz[b,e]oxepin-2-carboxylic acid, trans-11-(3-pyrrolidinopropylidene)-6,11-dihydrodibenz[b,e]oxepin-2-carboxylic acid, methyl cis-11-(4-dimethylaminobutylidene)-6,11-dihydrodibenz[b,e]oxepin-2-carboxylate, methyl trans-11-(4-dimethylaminobutylidene)-6,11-dihydrodibenz[b,e]oxepin-2-carboxylate, cis-11-(4-dimethylaminobutylidene)-6,11-dihydrodibenz[b,e]oxepin-2-carboxylic acid, trans-11-(4-dimethylaminobutylidene)-6,11-dihydrodibenz[b,e]oxepin-2-carboxylic acid, methyl cis-11-[2-(4-methylpiperazino)-ethylidene]-6,11-dihydrodibenz[b,e]oxepin-2-carboxylate, methyl trans-11-[2-(4-methylpiperazino)-ethylidene]-6,11-dihydrodibenz[b,e]oxepin-2-carboxylate, cis-11-[2-(4-methylpiperazino)ethylidene]-6,11-dihydrodibenz[b,e]oxepin-2-carboxylic acid, trans-11-[2-(4-methylpiperazino)ethylidene]-6,11-dihydrodibenz[b,e]oxepin-2-carboxylic acid, methyl cis-11-(2-morpholinoethylidene)-6,11-dihydrodibenz[b,e]oxepin-2-carboxylate, methyl trans-11-(2-morpholinoethylidene)-6,11-dihydrodibenz[b,e]oxepin-2-carboxylate, cis-11-(2-morpholinoethylidene)-6,11-dihydrodibenz[b,e]oxepin-2-carboxylic acid, trans-11-(2-morpholinoethylidene)-6,11-dihydrodibenz[b,e]oxepin-2-carboxylic acid, methyl cis-11-(2-thiomorpholinoethylidene)-6,11-dihydrodibenz[b,e]oxepin-2-carboxylate, methyl trans-11-(2-thiomorpholinoethylidene)-6,11-dihydrodibenz[b,e]oxepin-2-carboxylate, cis-11-(2-thiomorpholinoethylidene)-6,11-dihydrodibenz[b,e]oxepin-2-carboxylic acid, trans-11-(2-thiomorpholinoethylidene)-6,11-dihydrodibenz[b,e]oxepin-2-carboxylic acid, methyl cis-11-(2-pyrrolidinoethylidene)-6,11-dihydrodibenz[b,e]oxepin-2-carboxylate, methyl trans-11-(2-pyrrolidinoethylidene)-6,11-dihydrodibenz[b,e]oxepin-2-carboxylate, methyl cis-11-(2-piperidinoethylidene)-6,11-dihydrodibenz[b,e]oxepin-2-carboxylate, methyl trans-11-(2-piperidinoethylidene)-6,11-dihydrodibenz[b,e]oxepin-2-carboxylate, methyl cis-11-(3-dimethylaminopropylidene)-6,11-dihydrodibenz[b,e]oxepin-2-acetate, methyl trans-11-(3-dimethylaminopropylidene)-6,11-dihydrodibenz[b,e]oxepin-2-acetate, ethyl cis-11-(3-dimethylaminopropylidene)-6,11-dihydrodibenz[b,e]oxepin-2-acetate, ethyl trans-11-(3-dimethylaminopropylidene)-6,11-dihydrodibenz[b,e]oxepin-2-acetate, cis-11-(3-dimethylaminopropylidene)-6,11-dihydrodibenz[b,e]oxepin-2-acetic acid, trans-11-(3-dimethylaminopropylidene)-6,11-dihydrodibenz[b,e]oxepin-2-acetic acid, methyl cis-11-(4-dimethylaminobutylidene)-6,11-dihydrodibenz[b,e]oxepin-2-acetate, methyl trans-11-(4-dimethylaminobutylidene)-6,11-dihydrodibenz[b,e]oxepin-2-acetate, cis-11-(4-dimethylaminobutylidene)-6,11-dihydrodibenz[b,e]oxepin-2-acetic acid, trans-11-(4-dimethylaminobutylidene)-6,11-dihydrodibenz[b,e]oxepin-2-acetic acid, methyl cis-11-(3-pyrrolidinopropylidene)-6,11-dihydrodibenz[b,e]oxepin-2-acetate, methyl trans-11-(3-pyrrolidinopropylidene)-6,11-dihydrodibenz[b,e]oxepin-2-acetate, cis-11-(3-pyrrolidinopropylidene)-6,11-dihydrodibenz[b,e]oxepin-2-acetic acid, trans-11-(3-pyrrolidinopropylidene)-6,11-dihydrodibenz[b,e]oxepin-2-acetic acid, methyl cis-11-[2-(4-methylpiperazino)-ethylidene-6,11-dihydrodibenz[b,e]oxepin-2-acetate, methyl trans-11-[2-(4-methylpiperazino)-ethylidene)-6,11-dihydrodibenz[b,e]oxepin-2-acetate, cis-11-[2-(4-methylpiperazino)-ethylidene-6,11-dihydrodibenz[b,e]oxepin-2-acetic acid, trans-11-[2-(4-methylpiperazino)-ethylidene)-6,11-dihydrodibenz[b,e]oxepin-2-acetic acid, methyl cis-3-[1′-(3-dimethylaminopropylidene)-6,11-dihydrodibenz[b,e]oxepin-2-yl]-propionate, methyl trans-3-[11-(3-dimethylaminopropyli-dene)-6,11-dihydrodibenz[b,e]oxepin-2-yl]-propionate, cis-3-[11-(3-dimethylaminopropylidene)-6,11-dihydrodibenz[b,e]oxepin-2-yl]-propionic acid, trans-3-[11-(3-dimethylaminopropylidene)-6,11-dihydrodibenz[b,e]oxepin-2-yl]-propionic acid, methyl cis-11-(3-dimethylaminopropylidene)-6,11-dihydrodibenz[b,e]oxepin-3-acetate, methyl trans-11-(3-dimethylaminopropylidene)-6,11-dihydrodibenz[b,e]oxepin-3-acetate, cis-11-(3-dimethylaminopropylidene)-6,11-dihydrodibenz[b,e]oxepin-3-acetic acid, trans-11-(3-dimethylaminopropylidene)-6,11-dihydrodibenz[b,e]oxepin-3-acetic acid, cis-11-(3-dimethylaminopropylidene)-2-(2-hydroxyethyl)-6,11-dihydrodibenz[b,e]oxepin, trans-11-(3-dimethylaminopropylidene)-2-(2-hydroxyethyl)-6,11-dihydrodibenz[b,e]oxepin, cis-11-(3-dimethylaminopropylidene)-2-(2-triphenylmethyloxymethyl)-6,11-dihydrodibenz-[b,e]oxepin, trans-11-(3-dimethylaminopropylidene)-2-(2-triphenylmethyloxymethyl)-6,11-dihydrodibenz-[b,e]oxepin, cis-11-(3-dimethylaminopropylidene)-2-(3-hydroxypropyl)-6,11-dihydrodibenz[b,e]oxepin, trans-1-(3-dimethylaminopropylidene)-2-(3-hydroxypropyl)-6,11-dihydrodibenz[b,e]oxepin, methyl cis-11-(2-diethylaminoethyl)imino-6,11-dihydrodibenz[b,e]oxepin-2-carboxylate, methyl trans-11-(2-diethylaminoethyl)imino-6,11-dihydrodibenz[b,e]oxepin-2-carboxylate, cis-11-(2-diethylaminoethyl)imino-6,11-dihydrodibenz[b,e]oxepin-2-carboxylic acid, trans-11-(2-diethylaminoethyl)imino-6,11-dihydrodibenz[b,e]oxepin-2-carboxylic acid, methyl cis-11-(2-dimethylaminoethyl)imino-6,11-dihydrodibenz[b,e]oxepin-2-acetate, methyl trans-11-(2-dimethylaminoethyl)imino-6,11-dihydrodibenz[b,e]oxepin-2-acetate, cis-11-(2-dimethylaminoethyl)imino-6,11-dihydrodibenz[b,e]oxepin-2-acetic acid, trans-11-(2-dimethylaminoethyl)imino-6,11-dihydrodibenz[b,e]oxepin-2-acetic acid, methyl cis-11-(2-diethylaminoethyl)imino-6,11-dihydrodibenz[b,e]oxepin-2-acetate, methyl trans-11-(2-diethylaminoethyl)imino-6,11-dihydrodibenz[b,e]oxepin-2-acetate, cis-11-(2-diethylaminoethyl)imino-6,11-dihydrodibenz[b,e]oxepin-2-acetic acid, trans-11-(2-diethylaminoethyl)imino-6,11-dihydrodibenz[b,e]oxepin-2-acetic acid, methyl cis-11-(3-dimethylaminopropyl)imino-6,11-dihydrodibenz[b,e]oxepin-2-acetate, methyl trans-11-(3-dimethylaminopropyl)imino-6,11-dihydrodibenz[b,e]oxepin-2-acetate, cis-11-(3-dimethylaminopropyl)imino-6,11-dihydrodibenz[b,e]oxepin-2-acetic acid, trans-11-(3-dimethylaminopropyl)imino-6,11-dihydrodibenz[b,e]oxepin-2-acetic acid, methyl cis-3-[11-(2-diethylaminoethyl)imino-6,11-dihydrodibenz[b,e]oxepin-2-yl]-propionate, methyl trans-3-[11-(2-diethylaminoethyl)imino-6,11-dihydrodibenz[b,e]oxepin-2-yl]-propionate, cis-[11-(2-diethylaminoethyl)imino-6,11-dihydrodibenz[b,e]oxepin-2-yl]-propionic acid, trans-[11-(2-diethylaminoethyl)imino-6,11-dihydrodibenz[b,e]oxepin-2-yl]-propionic acid, methyl cis-2-[11-(2-dimethylaminoethyl)imino-6,11-dihydrodibenz[b,e]oxepin-2-yl]-propionate, methyl trans-2-[11-(2-dimethylaminoethyl)imino-6,11-dihydrodibenz[b,e]oxepin-2-yl]-propionate, cis-2-[11-(2-dimethylaminoethyl)imino-6,11-dihydrodibenz[b,e]oxepin-2-yl]-propionic acid, trans-2-[11-(2-dimethylaminoethyl)imino-6,11-dihydrodibenz[b,e]oxepin-2-yl]-propionic acid, methyl cis-11-(2-dimethylaminoethyl)imino-6,11-dihydrodibenz[b,e]oxepin-3-acetate, methyl trans-11-(2-dimethylaminoethyl)imino-6,11-dihydrodibenz[b,e]oxepin-3-acetate, cis-11-(2-dimethylaminoethyl)imino-6,11-dihydrodibenz[b,e]oxepin-3-acetic acid, trans-11-(2-dimethylaminoethyl)imino-6,11-dihydrodibenz[b,e]oxepin-3-acetic acid, methyl cis-11-(3-dimethylaminopropyl)imino-6,11-dihydrodibenz[b,e]oxepin-3-acetate, methyl trans-11-(3-dimethylaminopropyl)imino-6,11-dihydrodibenz[b,e]oxepin-3-acetate, cis-11-(3-dimethylaminopropyl)imino-6,11-dihydrodibenz[b,e]oxepin-3-acetic acid, trans-11-(3-dimethylaminopropyl)imino-6,11-dihydrodibenz[b,e]oxepin-3-acetic acid, methyl 11-(3-dimethylaminopropyl)-6,11-dihydrodibenz[b,e]oxepin-2-carboxylate, 11-(3-dimethylaminopropyl)-6,11-dihydrodibenz-[b,e]oxepin-2-carboxylic acid, 11-(3-dimethylaminopropyl)-6,11-dihydrodibenz-[b,e]oxepin-2-acetic acid, 11-(3-dimethylaminopropylidene)-2-(4,4-dimethyl-2-oxazoline-2-yl)-6,11-dihydrodibenz-[b,e]oxepin, 11-(3-dimethylaminopropyl)-2-(4,4-dimethyl-2-oxazoline-2-yl)-6,11-dihydrodibenz[b,e]oxepin, methyl cis-11-(3-morpholinopropylidene)-6,11-dihydrodibenz[b,e]oxepin-2-carboxylate, methyl trans-11-(3-morpholinopropylidene)-6,11-dihydrodibenz[b,e]oxepin-2-carboxylate, cis-11-(3-morpholinopropylidene)-6,11-dihydro-dibenz[b,e]oxepin-2-carboxylic acid, trans-11-(3-morpholinopropylidene)-6,11-dihydrodibenz[b,e]oxepin-2-carboxylic acid, methyl cis-11-(3-thiomorpholinopropylidene)-6,11-dihydrodibenz[b,e]oxepin-2-carboxylate, methyl trans-11-(3-thiomorpholinopropylidene)-6,11-dihydrodibenz[b,e]oxepin-2-carboxylate, cis-11-(3-thiomorpholinopropylidene)-6,11-dihydrodibenz[b,e]oxepin-2-carboxylic acid, trans-11-(3-thiomorpholinopropylidene)-6,11-dihydrodibenz[b,e]oxepin-2-carboxylic acid, methyl trans-3-[cis-11-(3-dimethylaminopro-pylidene)-6,11-dihydrodibenz[b,e]oxepin-2-yl]-acrylate, methyl trans-3-[trans-11-(3-dimethylaminopro-pylidene)-6,11-dihydrodibenz[b,e]oxepin-2-yl]-acrylate, trans-3-[cis-11-(3-dimethylaminopropylidene)-6,11-dihydrodibenz[b,e]oxepin-2-yl]-acrylic acid, trans-3-[trans-11-(3-dimethylaminopropylidene)-6,11-dihydrodibenz[b,e]oxepin-2-yl]-acrylic acid, methyl cis-11-(3-methylaminopropylidene)-6,11-dihydrodibenz[b,e]oxepin-2-acetate, methyl trans-11-(3-methylaminopropylidene)-6,11-dihydrodibenz[b,e]oxepin-2-acetate, cis-11-(3-methylaminopropylidene)-6,11-dihydrodibenz[b,e]oxepin-2-acetic acid, trans-11-(3-methylaminopropylidene)-6,11-dihydrodibenz[b,e]oxepin-2-acetic acid, methyl cis-11-(3-aminopropylidene)-6,11-dihydrodibenz[b,e]oxepin-2-acetate, methyl trans-11-(3-aminopropylidene)-6,11-dihydrodibenz[b,e]oxepin-2-acetate, cis-11-(3-aminopropylidene)-6,11-dihydrodibenz-[b,e]oxepin-2-acetic acid, methyl trans-11-(3-aminopropylidene)-6,11-dihydrodibenz[b,e]oxepin-2-acetic acid, or a salt, ester, or amide thereof.

In still further embodiments, the mast cell stabilizer is a spleen tyrosine kinase inhibitor. For example, the spleen tyrosine kinase inhibitor may be 2-[7-(3,4-dimethoxyphenyl)-imidazo[1,2-c]pyrimidin-5-ylamino]-nicotinamide dihydrochloride, 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide, NVP-QAB205, BAY 61-3606, piceatannol, 3,4-dimethyl-10-(3-aminopropyl)-9-acridone oxalate, a purine derivative, or a 1,6-naphthyridine derivative.

Other examples of mast cell stabilizers contemplated by the present invention include dihydropyridines. Exemplary dihydropyridines include nicardipine, barnidipine, YC-114, elgodipine, niguldipine and R(−)-niguldipine.

Administration of the pharmaceutically effective amount of the composition that comprises one or more mast cell stabilizers to a subject can be by any method known to those of ordinary skill in the art. For example, for treatment or prevention of an ophthalmic wound, administration can include topical application, subconjunctival injection, subtenon injection, periocular injection, retrobulbar injection, administration using a biodegradable insert (such as placement of a biodegradable insert or device onto the surface of or into ocular tissue), or administration using a medical device, such as a medical device coated with one or more mast cell stabilizers. In particular embodiments, the composition is formulated in an aqueous solution for topical application. Such formulations are discussed in detail in the specification below.

For topical application to the skin, administration can be by any method known to those of ordinary skill in the art. For example, the composition may be formulated in a cream, ointment, lotion, salve, solution, dispersion, or solution for topical application to the skin.

In some embodiments, the composition is applied directly onto a wound surface. In other embodiments, the composition is injected into a wound. One of ordinary skill in the art would be familiar with methods for applying a therapeutic composition to a skin or wound surface. Exemplary methods are discussed elsewhere in this specification.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Corneal epithelial defects are one of the most common ophthalmic conditions. Although many patients respond to conventional therapies, there remains a significant subset of patients who are refractory to conventional therapies. Thus, there is the need for more effective therapies of ocular surface wounds.

The inventors have identified novel methods of treating a wound in a subject that involve administering to the subject a pharmaceutically effective amount of a composition that includes a mast cell stabilizer, wherein administration of the composition results in treatment of the wound. More particularly, it has been found that topical administration of a mast cell stabilizer to an ophthalmic wound is a new and effective therapy for ocular surface wounds.

### A. Mast Cell Stabilizers

As discussed above, a “mast cell stabilizer” is a compound that inhibits the degranulation of sensitized and/or nonsensitized mast cells. One of ordinary skill in the art would be familiar with this class of agents. The mast cell stabilizers can either be obtained from commercial sources, or may be synthesized by methods known to those skilled in the art. Examples of mast cell stabilizers are discussed in greater detail below. Included as mast cell stabilizers are any enantiomers and pharmaceutically acceptable salts of any of the compounds set forth below. The compounds must be capable of stabilizing human connective tissue mast cells which contain both tryptase and chymase.

1. Olopatadine and Derivatives of Olopatadine

Olopatadine, 11-(3-dimethylaminopropylidene)-6,11-dihydrodibenz[b,e]oxepin-2-acetic acid is a known human conjunctival mast cell stabilizer and antihistamine which is used to treat allergic conjunctivitis. Unlike cromolyn and pemirolast, olopatadine inhibits histamine release from human conjunctival mast cells (Yanni et al., 1997). Olopatadine is disclosed in U.S. Pat. No. 5,116,863, the entire contents of which is herein incorporated by reference in its entirety. Use of olopatadine to treat ophthalmic allergic conditions is disclosed in U.S. Pat. No. 5,641,805, the entire contents of which is herein incorporated by reference in its entirety. Olopatadine includes the cis isomer, trans isomer, a mixture of cis and trans isomers, and pharmaceutically acceptable salts of olopatadine.

A “derivative of olopatadine” is defined herein to refer to any compound which is structurally and functionally similar to olopatadine. Included as derivatives of olopatadine are those structural variants set forth in U.S. Pat. No. 5,116,863, examples of which are set forth above.

Other derivatives of olopatadine include derivatives of doxepin that have mast cell stabilizing activity. U.S. Pat. Nos. 4,871,865 and 4,923,892 disclose certain carboxylic acid derivatives of doxepin that have such activity. U.S. Pat. Nos. 4,871,865 and 4,923,892 are each incorporated by reference in their entirety. In particular embodiments, the derivative of doxepin is 11-(3-dimethylaminopropylidene)-6,11-dihydrodibenz[b,e]oxepine-2-carboxylic acid, 11-(3-dimethylaminopropylidene)-6,11-dihydrodibenz[b,e]oxepine-2(E)-acrylic acid, or a salt, ester, or amide thereof.

Additional compounds considered to be derivatives of olopatadine in the context of the present invention include the imdazo[2,1-B]benzazepine derivatives set forth in U.S. Pat. No. 5,468,743, the entire contents of which is hereby specifically incorporated by references. Examples of such derivatives include 5,6-dihydro-11-(1-methyl-4-piperidinylidene)-11H-imidazo[2,1-b][3]benzazepine; 9-fluoro-6,11-dihydro-11-(1-methyl-4-piperidinylidene)-5H-imidazo[2,1-b][3]benzazepine; 11-(1-methyl-4-piperidinylidene)-11H-imidazo[2,1-b][3]benzazepine; 6,11-dihydro-11-(1-methyl-4-piperidinylidene)-5H-imidazo[2,1-b][3]benzazepine-3-methanol; 8-fluoro-6,11-dihydro-11-(1-methyl-4-piperidinylidene)-5H-imidazo[2,1-b][3]benzazepine; 6,11-dihydro-11-(1-methyl-4-piperidinylidene)-5H-imidazo[2,1-b][3]benzazepine-3-carboxaldehyde; 6,11-dihydro-11-(1-methyl-4-piperidinylidene)-5H-imidazo[2,1-b][3]benzazepine-3-carboxylic acid; 7-fluoro-6,11-dihydro-11-(1-methyl-4-piperidinylidene)-5H-imidazo[2,1-b][3]benzazepine; and 4-(8-fluoro-5,6-dihydro-1H-imidazo[2,1-b][3]benzazepin-11-ylidene)-1-piper idinepropanoic acid dihydrate.

2. Alcaftadine

Alcaftadine is 4-(1-methyl-piperidin-4-ylidene)-9,10-dihydro-4H-3,10a-diaza-benzo[f]azulene-1-carbaldehyde. The chemical structure of alcaftadine is as shown in formula (I):

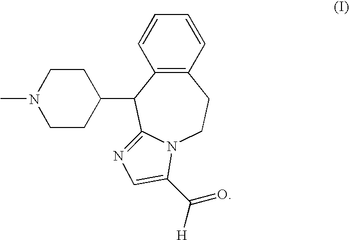

A “derivative of alcaftadine” is defined herein to refer to any compound which is structurally and functionally similar to alcaftadine. For example, the derivative of alcaftadine may be an imidazo[2,1-b][3]benzazepine of formula (II):

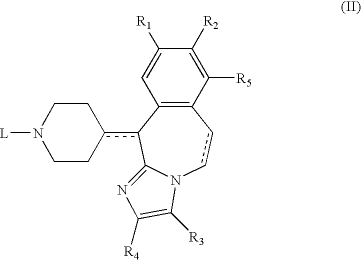

wherein each of the dotted lines independently represents an optional bond; R₁ represents hydrogen, halo, C₁₋₄ alkyl or C₁₋₄ alkyloxy; R₂ represents hydrogen, halo, C₁₋₄ alkyl or C₁₋₄ alkyloxy; R³ represents hydrogen, C₁₋₄ alkyl, ethenyl substituted with hydroxycarbonyl or C₁₋₄ alkyloxycarbonyl, C₁₋₄ alkyl substituted with hydroxycarbonyl or C₁₋₄ alkyloxycarbonyl, hydroxy C₁₋₄ alkyl, formyl or hydroxycarbonyl; R₄ represents hydrogen, C₁₋₄ alkyl, hydroxy C₁₋₄ alkyl, phenyl or halo; R₅ represents hydrogen, C₁₋₄ alkyl or halo; L represents hydrogen; C₁₋₆ alkyl; C₁₋₆ alkyl substituted with one substituent selected from the group consisting of hydroxy, halo, C₁₋₄ alkyloxy, hydroxycarbonyl, C₁₋₄ alkyloxycarbonyl, C₁₋₄ alkyloxycarbonyl-C₁₋₄ alkyloxy, hydroxycarbonyl C₁₋₄ alkyloxy, C₁₋₄ alkyloxycarbonylamino, C₁₋₄ alkylaminocarbonyl, C₁₋₄alkylaminocarbonylamino, C₁₋₄ alkylaminothiocarbonylamino, aryl, aryloxy and arylcarbonyl; C₁₋₆ alkyl substituted with both hydroxy and aryloxy; C₃₋₆ alkenyl; C₃₋₆ alkenyl substituted with aryl, or a pharmaceutically acceptable salt or stereochemical isomer thereof. These and other structural variants of alcaftadine are set forth in U.S. Pat. No. 5,468,743, which is herein specifically incorporated by reference in its entirety.

In particular embodiments, the derivative of alcaftadine is 4-(1-methyl-piperidin-4-ylidene)-9,10-dihydro-4H-3,10a-diaza-benzo[f]azulene, which has the chemical structure shown in formula (III):

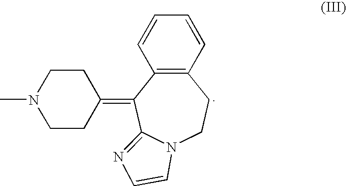

As used above for Figure (II), halo refers to fluoro, chloro, bromo and iodo; C₁₋₄ alkyl refers to straight and branched chain saturated hydrocarbon radicals having from 1 to 4 carbon atoms such as, for example, methyl, ethyl, propyl, 1-methylethyl, butyl, 1-methylpropyl, 2-methylpropyl and 1,1-dimethylethyl; C₁₋₆ alkyl refers to C₁₋₄ alkyl radicals as defined herein and the higher homologs thereof having from 5 to 6 carbon atoms such as, for example, pentyl and hexyl; C₃₋₆ alkenyl refers to straight and branched chain hydrocarbon radicals containing one double bond and having from 3 to 6 carbon atoms such as, for example, 2-propenyl, 2-butenyl, 3-butenyl, 2-methyl-2-propenyl, 2-pentenyl, 3-pentenyl, 3,3-dimethyl-2-propenyl, hexenyl and the like; C₁₋₄ alkanediyl refers to bivalent straight or branched chain hydrocarbon radicals containing from 1 to 4 carbon atoms such as, for example, methylene, 1,1-ethanediyl, 1,2-ethanediyl, 1,3-propanediyl, 1,4-butanediyl and the like.

The term “pharmaceutically acceptable salt” or “pharmaceutically acceptable addition salt” as used herein refers to the nontoxic, therapeutically active addition salt forms which the compounds of formula (II) may form. The compounds of formula (II) having basic properties may be converted into the corresponding therapeutically active, non-toxic acid addition salt forms by treating the free base form with a suitable amount of an appropriate acid following conventional procedures. Examples of appropriate acids are for example, inorganic acids, for example, hydrohalic acid, e.g., hydrochloric, hydrobromic and the like acids, sulfuric acid, nitric acid, phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids.

The compounds of formula (II) having acidic properties may be converted in a similar manner into the corresponding therapeutically active, non-toxic base addition salt forms. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g., arginine, lysine. The term “pharmaceutically acceptable addition salts” also comprises the solvates which the compounds of formula (II) may form, e.g., the hydrates, alcoholates and the like.

The term “stereochemically isomeric form” or “stereochemical isomer” as used herein refers to the possible different isomeric as well as conformational forms which the compounds of formula (II) may possess. Unless otherwise mentioned or indicated, the chemical designation of compounds denotes the mixture of all possible stereochemically and conformationally isomeric forms, said mixtures containing all diastereomers, enantiomers, and/or conformers of the basic molecular structure. All stereochemically isomeric forms of the compounds of formula (II) both in pure form or in admixture with each other are intended to be embraced within the scope of the present invention.

Some compounds of the present invention may exist in different tautomeric forms and all such tautomeric forms are intended to be included within the scope of the present invention.

3. Dihydropyridines

U.S. Pat. No. 6,225,327, herein specifically incorporated by reference in its entirety, discloses dihydropyridines that are known to inhibit human conjunctival mast cell degranulation. Each of the compounds set forth therein is contemplated as a mast cell stabilizer. Exemplary dihydropyridines include nicardipine, barnidipine, YC-114, elgodipine, niguldipine and R(−)-niguldipine.

4. Spleen Tyrosine Kinase Inhibitors

Spleen tyrosine kinase (Syk) is a protein tyrosine kinase that plays a pivotal role in high affinity IgE receptor signaling in mast cells. Syk is also involved in antigen receptor signaling of B and T lymphocytes and in eosinophil survival in response to IL-5 and GM-CSF. Syk has been implicated as playing a functional role in mast cell development and IgE-mediated allergen-induced airway hyperresponsiveness.

Peptide inhibitors of Syk kinase are discussed in U.S. Pat. No. 5,858,981, hereby specifically incorporated by reference in its entirety. Examples of such peptide inhibitors include peptides comprising the sequence YXXL (SEQ ID NO:1), wherein XX represents any two amino acids. For example, the YXXL sequence can be a YXXL sequence of the cytoplasmic domain of FcγRIIA. Alternatively the Syk inhibitor can be an antisense oligonucleotide, such as any of the antisense oligonucleotides set forth in U.S. Pat. No. 5,858,981, hereby specifically incorporated by reference in its entirety. Examples of such antisense oligonucleotides include SEQ ID NO:2, SEQ ID NO:3, SEQ ID NO:4, and SEQ ID NO:5 (set forth in U.S. Pat. No. 5,858,981 as SEQ ID NOs:21-24, respectively).

Another inhibitor of Syk is 2-[7-(3,4-dimethoxyphenyl)-imidazo[1,2-c]pyrimidin-5-ylamino]-nicotinamide dihydrochloride (Yamamoto et al., 2003). Other inhibitors of Syk include small molecules such as indole derivatives and isoindole derivatives (Cox et al., 2003).

Pyrimidine-5-carboxamide derivatives such as those described in WO 99/31073, hereby incorporated by reference herein, are also included as inhibitors of Syk. Examples include 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide.

Purine derivative inhibitors of Syk are also contemplated, and these are discussed in greater detail in U.S. Pat. No. 6,589,950, herein specifically incorporated by reference in its entirety. U.S. Patent App. Pub. No. 20030229090, herein specifically incorporated by reference in its entirety, describes 1,6-naphthyridine derivatives that are Syk inhibitors. Small interfering RNA (siRNA) that inhibit Syk expression are also contemplated, as described in U.S. Patent App. Pub. Nos. 20050075306 and 20050267059, each of which is herein specifically incorporated by reference in its entirety.

Other Syk inhibitors that can be applied in the context of the present invention include NVP-QAB205, BAY 61-3606, piceatannol, and 3,4-dimethyl-10-(3-aminopropyl)-9-acridone oxalate).

One of ordinary skill in the art would be able to identify additional inhibitors of Syk. Methods of identifying modulators of the Syk family of tyrosine kinases are discussed in U.S. Patent App. Pub. No. 20030113828, herein specifically incorporated by reference in its entirety.

### B. Treatment of Disease

1. Definitions

“Treatment” and “treating” refer to administration or application of a therapeutic agent to a subject or performance of a procedure or modality on a subject for the purpose of obtaining a therapeutic benefit of a disease or health-related condition. For example, in the context of the present invention, a corneal abrasion may be treated by topically applying to the ocular surface a pharmaceutically effective amount of a mast cell stabilizer for the purpose of facilitating the restoration of the integrity of the corneal epithelium.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, regarding the treatment of a cornal abrasion, a therapeutic benefit is obtained when there is decreased associated pain or decrease in size of the corneal abrasion.

A “disease” or “health-related condition” can be any pathological condition of a body part, an organ, or a system resulting from any cause, such as infection, genetic defect, and/or environmental stress. The cause may or may not be known. Examples of a disease or health related condition include ophthalmic conditions such as dry eye syndrome, meibomitis, conjunctivitis, iritis, age-related macular degeneration, glaucoma, a corneal laceration, or a traumatic corneal abrasion.

The subject can be a subject who is known or suspected of being free of a particular disease or health-related condition at the time the relevant preventive agent is administered. The subject, for example, can be a subject with no known disease or health-related condition (i.e., a healthy subject). In some embodiments, the subject is a subject at risk of developing a particular disease or health-related condition. For example, the subject may be a subject with a history of recurrent corneal erosion of unknown etiology who currently has no corneal epithelial defect but who is at risk of developing a corneal erosion.

In additional embodiments of the invention, methods include identifying a patient in need of treatment. A patient may be identified, for example, based on taking a patient history, or based on findings on clinical examination.

### C. Pharamaceutics and Formulations

1. Dosage

The phrase “pharmaceutically effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to promote the healing of a wound, such as an ophthalmic wound. The effective amount may vary depending on such factors as the disease or condition being treated, the particular composition being administered, or the severity of the disease or condition.

The phrase “pharmaceutically acceptable” is art-recognized and refers to compositions, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art.

The amount of drug to be included in the compositions or applied in the methods set forth herein will be whatever amount is pharmaceutically effective and will depend upon a number of factors, including the identity and potency of the chosen drug. One of ordinary skill in the art would be familiar with factors that are involved in determining a pharmaceutically effective dose of a drug.

In particular embodiments, the composition is administered once a day. However, the compositions of the present invention may also be formulated for administration at any frequency of administration, including once a week, once every 5 day, once every 3 days, once every 2 days, twice a day, three times a day, four times a day, five times a day, six times a day, eight times a day, every hour, or any greater frequency. One of ordinary skill in the art would be familiar with establishing a therapeutic regimen. Factors involved in this determination include the disease to be treated, particular characteristics of the subject, and the particular formulation of mast cell stabilizer.

2. Formulations

Regarding the methods set forth herein, compositions of mast cell stabilizers can be formulated in any manner known to those of ordinary skill in the art. For example, for application to a skin surface or skin wound, the composition may be formulated as a liquid, a cream, an ointment, a gel, a solution, a dispersion, a rinse, and so forth.

In the compositions set forth herein, the concentration of the mast cell stabilizer can be any concentration known or suspected by those of ordinary skill in the art to be of benefit in the treatment or prevention of a wound. Unless indicated otherwise, all component amounts are presented on a % (w/v) basis.

In particular embodiments, the total concentration of the mast cell stabilizer is about 10% or less. In more particular embodiments, the total concentration of the mast cell stabilizer is about 5% or less. In certain embodiments, the concentration of the mast cell stabilizer in the composition is in the range of about 0.001 to about 10%. In further embodiments, the concentration of the mast cell stabilizer is in the range of about 0.05% to about 5%. In still further embodiments, the concentration of mast cell stabilizer in the composition is in the range of about 0.0025% to about 1%. For example, the concentration of the mast cell stabilizer may be in the range of about 0.03% to about 3.0%, about 0.05% to about 2%, about 0.1% to about 1.5%, about 0.2% to about 1.0%, about 0.3% to about 0.75%, any concentration or range derivable therein. In particular embodiments, the concentration of mast cell stabilizer is about 0.075% to about 0.125%.

In certain embodiments of the present invention, the compositions set forth herein include more than one mast cell stabilizer. One of ordinary skill in the art would be familiar with preparing and administering pharmaceutical compositions that include more than one therapeutic agent. In some embodiments, the composition includes one or more additional therapeutic agents that are not mast cell stabilizers.

In addition to the mast cell stabilizer, the compositions of the present invention optionally comprise one or more excipients. Excipients commonly used in pharmaceutical compositions include, but are not limited to, carriers, tonicity agents, preservatives, chelating agents, buffering agents, surfactants and antioxidants.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient.

Any of a variety of carriers may be used in the formulations of the present invention including water, mixtures of water and water-miscible solvents, such as C1-C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, mixtures of those polymers. The concentration of the carrier is, typically, from 1 to 100,000 times the concentration of the active ingredient.

Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable antioxidants include sulfites, ascorbates, BHA and BHT. The compositions of the present invention optionally comprise an additional active agent.

In particular embodiments, the compositions are suitable for application to mammalian eyes. For example, for ophthalmic administration, the formulation may be a solution, a suspension, a gel, or an ointment. In some embodiments, the composition is administered via a bioerodible implant.

In preferred aspects, the compositions that include mast cell stabilizers will be formulated for topical application to the eye in aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

The compositions of the present invention are preferably not formulated as solutions that undergo a phase transition to a gel upon administration to the eye.

In addition to the one or more mast cell stabilizers, the compositions of the present invention may contain other ingredients as excipients. For example, the compositions may include one or more pharmaceutically acceptable buffering agents, preservatives (including preservative adjuncts), non-ionic tonicity-adjusting agents, surfactants, solubilizing agents, stabilizing agents, comfort-enhancing agents, polymers, emollients, pH-adjusting agents and/or lubricants.

For topical formulations to the eye, the formulations are preferably isotonic, or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of 5-9, preferably 6.5-7.5, and most preferably 6.9-7.4.

The formulations set forth herein may comprise one or more preservatives. Examples of preservatives include quaternary ammonium compounds, such as benzalkonium chloride or benzoxonium chloride. Other examples of preservatives include sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, or sorbic acid.

In certain embodiments, the mast cell stabilizer is formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. The formulation of the present invention may be used with contact lenses or other ophthalmic products.

In particular embodiments of the present invention, the method involves administration to a subject of a pharmaceutically effective amount of a composition that includes olopatadine or a derivative of olopatadine.

Topical olopatadine formulations that have prolonged therapeutic activity and are effective as products for treating allergic or inflammatory conditions in the eye and nose are desirable. Topical olopatadine formulations that are effective as once-a-day products for treating allergic conditions in the eye are desirable. PATANOL® (olopatadine hydrochloride ophthalmic solution) 0.1% is currently the only commercially available olopatadine product for ophthalmic use. According to its to labeling information, it contains olopatadine hydrochloride equivalent to 0.1% olopatadine, 0.01% benzalkonium chloride, and unspecified amounts of sodium chloride, dibasic sodium phosphate, hydrochloric acid and/or sodium hydroxide (to adjust pH) and purified water.

In particular embodiments, the concentration of olopatadine in the compositions of the present invention will range from 0.01% to 0.8%, and is preferably from 0.1-0.8%. Olopatadine is preferably added in the form of olopatadine hydrochloride.

Generally, olopatadine will be added in the form of a pharmaceutically acceptable salt. Examples of the pharmaceutically acceptable salts of olopatadine include inorganic acid salts such as hydrochloride, hydrobromide, sulfate and phosphate; organic acid salts such as acetate, maleate, fumarate, tartrate and citrate; alkali metal salts such as sodium salt and potassium salt; alkaline earth metal salts such as magnesium salt and calcium salt; metal salts such as aluminum salt and zinc salt; and organic amine addition salts such as triethylamine addition salt (also known as tromethamine), morpholine addition salt and piperidine addition salt. The most preferred form of olopatadine for use in the solution compositions of the present invention is the hydrochloride salt of (Z)-1-(3-dimethylaminopropylidene)-6,11-dihydro-dibenz-[b,e]oxepin-2-acetic acid. When olopatadine is added to the compositions of the present invention in this salt form, 0.222% olopatadine hydrochloride is equivalent to 0.2% olopatadine free base, 0.443% olopatadine hydrochloride is equivalent to 0.4% olopatadine free base, and 0.665% olopatadine hydrochloride is equivalent to 0.6% olopatadine free base.

In addition to olopatadine, the aqueous solution compositions of the present invention may comprise polyvinylpyrrolidone or polystyrene sulfonic acid in an amount sufficient to enhance the physical stability of the composition. Polyvinylpyrrolidone and polystyrene sulfonic acid are known polymers and both are commercially available from a variety of sources in different grades and in a number of molecular weights. The amount of polyvinylpyrrolidone contained in the compositions of the present invention may be 0.1-3%, preferably 0.2-2%, and most preferably 1.5-2%.

Polystyrene sulfonic acid is commercially available in many grades. In general, the amount of polystyrene sulfonic acid contained in the compositions of the present invention will be 0.1-1%. In particular embodiments, it is 0.15-0.4%, and in more particular embodiments, it is 0.25%.

The compositions of the present invention comprise 0.17-0.62% olopatadine and a polymeric physical stability-enhancing ingredient consisting essentially of polyvinylpyrrolidone or polystyrene sulfonic acid in an amount sufficient to enhance the physical stability of the solution.

In some embodiments, the compositions set forth herein have a viscosity of 0.5-10 cps, preferably 0.5-5 cps, and most preferably 1-2 cps. This relatively low viscosity insures that the product is comfortable, does not cause blurring, and is easily processed during manufacturing, transfer and filling operations.

3. Route of Administration

In the methods set forth herein, administration to a subject of a pharmaceutically effective amount of a composition that includes one or more mast cell stabilizers may be by any method known to those of ordinary skill in the art.

For example, the composition may be administered locally, topically, intradermally, intralesionally, to the surface of a wound, topically, intratumorally, subcutaneously, subconjunctival, mucosally, by injection, by infusion, by continuous infusion, by localized perfusion bathing target cells directly, via a catheter, or via a lavage.

In particular embodiments, the composition is administered topically to the ocular surface, such as to the surface of an epithelial wound. In further particular embodiments, the composition is administered topically to a skin surface or the surface of a skin wound. Regarding ophthalmic administration, it is contemplated that all local routes to the eye may be used. Most preferably, the ophthalmic administration is topical.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preferred Topical Ophthalmic Solution Formulation

| Ingredient | Concentration (W/V %) |
|---|---|
| Human connective tissue mast cell stabilizer | 0.0025-1.0 |
| Dibasic Sodium Phosphate | 0.5 |
| (Anhydrous), USP | |
| Sodium Chloride, USP | 0.65 |
| Benzalkonium Chloride | 0.01 |
| Sodium Hydroxide, NF | q.s. pH 6.8-7.4 |
| Hydrochloric Acid, NF | q.s. pH 6.8-7.4 |
| Purified Water | q.s. 100 |

### Example 2

### Topical Ophthalmic Gel Formulation

| Ingredient | Concentration (W/V %) |
|---|---|
| Human connective tissue mast cell stabilizer | 0.0025-1.0 |
| Carbopol 974 P | 0.8 |
| Disodium EDTA | 0.01 |
| Polysorbate 80 | 0.05 |
| Benzalkonium Chloride, Solution | 0.01 + 5% excess |
| Sodium Hydroxide | q.s. pH 6.8-7.4 |
| Hydrochloric acid | q.s. pH 6.8-7.4 |
| Water for Injection | q.s. 100 |

All of the methods disclosed and claimed herein can be executed without undue experimentation in light of the present disclosure. While the methods of this invention have been described in terms of preferred embodiments, it will be apparent to those of skill in the art that variations may be applied in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,871,865
U.S. Pat. No. 4,923,892
U.S. Pat. No. 5,116,863
U.S. Pat. No. 5,468,743
U.S. Pat. No. 5,641,805
U.S. Pat. No. 5,858,981
U.S. Pat. No. 6,225,327
U.S. Pat. No. 6,589,950
U.S. Publn. 20030229090
U.S. Publn. 20030113828
U.S. Publn. 20050075306
U.S. Publn. 20050267059
Buckley et al., J. Pathol., 189(1):138-143, 1999.
Cox et al., In: Heterocylic Compounds, Aventis Pharm., NJ, 34(51), 2003.
Gordon, Curr. Eye Res., 30(5):385-394, 2005.
Nishikori et al., Arch. Dermatol. Res., 290(10):553-560, 1998.
PCT Appln. WO 99/31073
Yamamoto et al., J. Pharmacol. Exp. Ther., 306(3):1174-1181, 2003.
Yanni et al., Ann. Allergy Asthma Immunol., 79(6):541-545, 1997.

What is claimed is:

1. A method of treating an ophthalmic or dermal wound in a subject, comprising administering to the subject a pharmaceutically effective amount of a composition comprising a human connective tissue mast cell stabilizer.

2. The method of claim 1, wherein the subject is a human.

3. The method of claim 1, wherein the ophthalmic wound is a conjunctival wound.

4. The method of claim 1, wherein the ophthalmic wound is a corneal wound.

5. The method of claim 4, wherein the corneal wound is a corneal epithelial defect, a recurrent corneal erosion, or a corneal ulcer.

6. The method of claim 5, wherein the corneal wound is a corneal epithelial defect is further defined as a corneal epithelial defect secondary to dry eye syndrome, allergic conjunctivitis, giant papillary conjunctivitis, vernal conjunctivitis, atopic keratoconjunctivitis, viral keratitis, bacterial keratitis, trauma, ophthalmic surgery, chemical exposure, exposure keratopathy, or an unknown etiology.

7. The method of claim 1, wherein the wound is a dermal wound.

8. The method of claim 7, wherein the dermal wound is a traumatic wound, a surgical wound, a wound secondary to an infection, or a burn.

9. The method of claim 7, wherein the wound is a dermal abrasion.

10. The method of claim 1, wherein administering is further defined as topical application.

11. The method of claim 1, wherein the mast cell stabilizer is olopatadine, a derivative of olopatadine, alcaftadine, a derivative of alcaftadine, a spleen tyrosine kinase inhibitor, or a dihydropyridine.

12. The method of claim 11, wherein the mast cell stabilizer is olopatadine.

13. The method of claim 11, wherein the mast cell stabilizer is a derivative of olopatadine.

14. The method of claim 13, wherein the derivative of olopatadine is (Z)-11-(3-(dimethylamino)propylidene)-6,11-dihydrodibenz[b,e]oxepin-2-carboxylic acid, (E)-11-(3-(dimethylamino)propylidene)-6,11-dihydrodibenz[b,e]oxepin-2-carboxylic acid, (E)-11-(3-(dimethylamino)propylidene)-6,11-dihydrodibenz[b,e]oxepin-3-carboxylic acid, (Z)-11-(3-(dimethylamino)propylidene)-6,11-dihydrodibenz[b,e]oxepin-3-carboxylic acid, (E)-11-(3-(dimethylamino)propylidene)-6,11-dihydrodibenz[b,e]oxepin-8-carboxylic acid, (Z)-11-(3-(dimethylamino)propylidene)-6,11-dihydrodibenz[b,e]oxepin-8-carboxylic acid, (E)-11-(3-(dimethylamino)propylidene)-6,11-dihydrodibenz[b,e]oxepin-9-carboxylic acid, (Z)-11-(3-(dimethylamino)propylidene)-6,11-dihydrodibenz[b,e]oxepin-9-carboxylic acid, (E)-11-(3-(dimethylamino)propylidene)-6,11-dihydrodibenz[b,e]oxepin-2-acrylic acid, (Z)-11-(3-(dimethylamino)propylidene)-6,11-dihydrodibenz[b,e]oxepin-2-acrylic acid, (E)-5-(3-(dimethylamino)propylidene)-10,11-dihydro-5H-dibenzo[a,d]cyclohepten-3-carboxylic acid, and (Z)-5-(3-(dimethylamino)propylidene)-10,11-dihydro-5H-dibenzo[a,d]cyclohepten-3-carboxylic acid, 11-(3-dimethylaminopropylidene)-6,11-dihydrodibenz[b,e]oxepine-2-carboxylic acid, 11-(3-dimethylaminopropylidene)-6,11-dihydrodibenz[b,e]oxepine-2(E)-acrylic acid, 5,6-dihydro-11-(1-methyl-4-piperidinylidene)-1H-imidazo[2,1-b][3]benzazepine, 9-fluoro-6,11-dihydro-11-(1-methyl-4-piperidinylidene)-5H-imidazo[2,1-b][3]benzazepine, 11-(1-methyl-4-piperidinylidene)-1H-imidazo[2,1-b][3]benzazepine, 6,11-dihydro-11-(1-methyl-4-piperidinylidene)-5H-imidazo[2,1-b][3]benzazepine-3-methanol, 8-fluoro-6,11-dihydro-11-(1-methyl-4-piperidinylidene)-5H-imidazo[2,1-b][3]benzazepine, 6,11-dihydro-11-(1-methyl-4-piperidinylidene)-5H-imidazo[2,1-b][3]benzazepine-3-carboxaldehyde, 6,11-dihydro-11-(1-methyl-4-piperidinylidene)-5H-imidazo[2,1-b][3]benzazepine-3-carboxylic acid, 7-fluoro-6,11-dihydro-11-(1-methyl-4-piperidinylidene)-5H-imidazo[2,1-b][3]benzazepine, 4-(8-fluoro-5,6-dihydro-11H-imidazo[2,1-b][3]benzazepin-11-ylidene)-1-piper idinepropanoic acid dihydrate, methyl cis-11-(3-dimethylaminopropylidene)-6,11-dihydrodibenz[b,e]oxepin-2-carboxylate, methyl trans-11-(3-dimethylaminopropylidene)-6,11-dihydrodibenz[b,e]oxepin-2-carboxylate, ethyl cis-11-(3-dimethylaminopropylidene)-6,11-dihydrodibenz[b,e]oxepin-2-carboxylate, ethyl trans-11-(3-dimethylaminopropylidene)-6,11-dihydrodibenz[b,e]oxepin-2-carboxylate, cis-11-(3-dimethylaminopropylidene)-6,11-dihydrodibenz[b,e]oxepin-2-carboxylic acid, trans-11-(3-dimethylaminopropylidene)-6,11-dihydrodibenz[b,e]oxepin-2-carboxylic acid, methyl cis-11-(3-diethylaminopropylidene)-6,11-dihydrodibenz[b,e]oxepin-2-carboxylate, methyl trans-11-(3-diethylaminopropylidene)-6,11-dihydrodibenz[b,e]oxepin-2-carboxylate, cis-11-(3-diethylaminopropylidene)-6,11-dihydrodibenz[b,e]oxepin-2-carboxylic acid, trans-11-(3-diethylaminopropylidene)-6,11-dihydrodibenz[b,e]oxepin-2-carboxylic acid, methyl cis-11-(3-pyrrolidinopropylidene)-6,11-dihydrodibenz[b,e]oxepin-2-carboxylate, methyl trans-11-(3-pyrrolidinopropylidene)-6,11-dihydrodibenz[b,e]oxepin-2-carboxylate, cis-11-(3-pyrrolidinopropylidene)-6,11-dihydrodibenz[b,e]oxepin-2-carboxylic acid, trans-11-(3-pyrrolidinopropylidene)-6,11-dihydrodibenz[b,e]oxepin-2-carboxylic acid, methyl cis-11-(4-dimethylaminobutylidene)-6,11-dihydrodibenz[b,e]oxepin-2-carboxylate, methyl trans-11-(4-dimethylaminobutylidene)-6,11-dihydrodibenz[b,e]oxepin-2-carboxylate, cis-11-(4-dimethylaminobutylidene)-6,11-dihydrodibenz[b,e]oxepin-2-carboxylic acid, trans-11-(4-dimethylaminobutylidene)-6,11-dihydrodibenz[b,e]oxepin-2-carboxylic acid, methyl cis-11-[2-(4-methylpiperazino)-ethylidene]-6,11-dihydrodibenz[b,e]oxepin-2-carboxylate, methyl trans-11-[2-(4-methylpiperazino)-ethylidene]-6,11-dihydrodibenz[b,e]oxepin-2-carboxylate, cis-11-[2-(4-methylpiperazino)ethylidene]-6,11-dihydrodibenz[b,e]oxepin-2-carboxylic acid, trans-11-[2-(4-methylpiperazino)ethylidene]-6,11-dihydrodibenz[b,e]oxepin-2-carboxylic acid, methyl cis-11-(2-morpholinoethylidene)-6,11-dihydrodibenz[b,e]oxepin-2-carboxylate, methyl trans-11-(2-morpholinoethylidene)-6,11-dihydrodibenz[b,e]oxepin-2-carboxylate, cis-11-(2-morpholinoethylidene)-6,11-dihydrodibenz[b,e]oxepin-2-carboxylic acid, trans-11-(2-morpholinoethylidene)-6,11-dihydrodibenz[b,e]oxepin-2-carboxylic acid, methyl cis-11-(2-thiomorpholinoethylidene)-6,11-dihydrodibenz[b,e]oxepin-2-carboxylate, methyl trans-11-(2-thiomorpholinoethylidene)-6,11-dihydrodibenz[b,e]oxepin-2-carboxylate, cis-11-(2-thiomorpholinoethylidene)-6,11-dihydrodibenz[b,e]oxepin-2-carboxylic acid, trans-11-(2-thiomorpholinoethylidene)-6,11-dihydrodibenz[b,e]oxepin-2-carboxylic acid, methyl cis-11-(2-pyrrolidinoethylidene)-6,11-dihydrodibenz[b,e]oxepin-2-carboxylate, methyl trans-11-(2-pyrrolidinoethylidene)-6,11-dihydrodibenz[b,e]oxepin-2-carboxylate, methyl cis-11-(2-piperidinoethylidene)-6,11-dihydrodibenz[b,e]oxepin-2-carboxylate, methyl trans-11-(2-piperidinoethylidene)-6,11-dihydrodibenz[b,e]oxepin-2-carboxylate, methyl cis-11-(3-dimethylaminopropylidene)-6,11-dihydrodibenz[b,e]oxepin-2-acetate, methyl trans-11-(3-dimethylaminopropylidene)-6,11-dihydrodibenz[b,e]oxepin-2-acetate, ethyl cis-11-(3-dimethylaminopropylidene)-6,11-dihydrodibenz[b,e]oxepin-2-acetate, ethyl trans-11-(3-dimethylaminopropylidene)-6,11-dihydrodibenz[b,e]oxepin-2-acetate, cis-11-(3-dimethylaminopropylidene)-6,11-dihydrodibenz[b,e]oxepin-2-acetic acid, trans-11-(3-dimethylaminopropylidene)-6,11-dihydrodibenz[b,e]oxepin-2-acetic acid, methyl cis-11-(4-dimethylaminobutylidene)-6,11-dihydrodibenz[b,e]oxepin-2-acetate, methyl trans-11-(4-dimethylaminobutylidene)-6,11-dihydrodibenz[b,e]oxepin-2-acetate, cis-11-(4-dimethylaminobutylidene)-6,11-dihydrodibenz[b,e]oxepin-2-acetic acid, trans-11-(4-dimethylaminobutylidene)-6,11-dihydrodibenz[b,e]oxepin-2-acetic acid, methyl cis-11-(3-pyrrolidinopropylidene)-6,11-dihydrodibenz[b,e]oxepin-2-acetate, methyl trans-11-(3-pyrrolidinopropylidene)-6,11-dihydrodibenz[b,e]oxepin-2-acetate, cis-11-(3-pyrrolidinopropylidene)-6,11-dihydrodibenz[b,e]oxepin-2-acetic acid, trans-11-(3-pyrrolidinopropylidene)-6,11-dihydrodibenz[b,e]oxepin-2-acetic acid, methyl cis-11-[2-(4-methylpiperazino)-ethylidene-6,11-dihydrodibenz[b,e]oxepin-2-acetate, methyl trans-11-[2-(4-methylpiperazino)-ethylidene)-6,11-dihydrodibenz[b,e]oxepin-2-acetate, cis-11-[2-(4-methylpiperazino)-ethylidene-6,11-dihydrodibenz[b,e]oxepin-2-acetic acid, trans-11-[2-(4-methylpiperazino)-ethylidene)-6,11-dihydrodibenz[b,e]oxepin-2-acetic acid, methyl cis-3-[11-(3-dimethylaminopropylidene)-6,11-dihydrodibenz[b,e]oxepin-2-yl]-propionate, methyl trans-3-[11-(3-dimethylaminopropyli-dene)-6,11-dihydrodibenz[b,e]oxepin-2-yl]-propionate, cis-3-[11-(3-dimethylaminopropylidene)-6,1-dihydrodibenz[b,e]oxepin-2-yl]-propionic acid, trans-3-[11-(3-dimethylaminopropylidene)-6,11-dihydrodibenz[b,e]oxepin-2-yl]-propionic acid, methyl cis-11-(3-dimethylaminopropylidene)-6,11-dihydrodibenz[b,e]oxepin-3-acetate, methyl trans-11-(3-dimethylaminopropylidene)-6,11-dihydrodibenz[b,e]oxepin-3-acetate, cis-11-(3-dimethylaminopropylidene)-6,11-dihydrodibenz[b,e]oxepin-3-acetic acid, trans-11-(3-dimethylaminopropylidene)-6,11-dihydrodibenz[b,e]oxepin-3-acetic acid, cis-11-(3-dimethylaminopropylidene)-2-(2-hydroxyethyl)-6,11-dihydrodibenz[b,e]oxepin, trans-11-(3-dimethylaminopropylidene)-2-(2-hydroxyethyl)-6,11-dihydrodibenz[b,e]oxepin, cis-11-(3-dimethylaminopropylidene)-2-(2-triphenylmethyloxymethyl)-6,11-dihydrodibenz-[b,e]oxepin, trans-11-(3-dimethylaminopropylidene)-2-(2-triphenylmethyloxymethyl)-6,11-dihydrodibenz-[b,e]oxepin, cis-11-(3-dimethylaminopropylidene)-2-(3-hydroxypropyl)-6,11-dihydrodibenz[b,e]oxepin, trans-11-(3-dimethylaminopropylidene)-2-(3-hydroxypropyl)-6,11-dihydrodibenz[b,e]oxepin, methyl cis-11-(2-diethylaminoethyl)imino-6,11-dihydrodibenz[b,e]oxepin-2-carboxylate, methyl trans-11-(2-diethylaminoethyl)imino-6,11-dihydrodibenz[b,e]oxepin-2-carboxylate, cis-11-(2-diethylaminoethyl)imino-6,11-dihydrodibenz[b,e]oxepin-2-carboxylic acid, trans-11-(2-diethylaminoethyl)imino-6,11-dihydrodibenz[b,e]oxepin-2-carboxylic acid, methyl cis-11-(2-dimethylaminoethyl)imino-6,11-dihydrodibenz[b,e]oxepin-2-acetate, methyl trans-11-(2-dimethylaminoethyl)imino-6,11-dihydrodibenz[b,e]oxepin-2-acetate, cis-11-(2-dimethylaminoethyl)imino-6,11-dihydrodibenz[b,e]oxepin-2-acetic acid, trans-11-(2-dimethylaminoethyl)imino-6,11-dihydrodibenz[b,e]oxepin-2-acetic acid, methyl cis-11-(2-diethylaminoethyl)imino-6,11-dihydrodibenz[b,e]oxepin-2-acetate, methyl trans-11-(2-diethylaminoethyl)imino-6,11-dihydrodibenz[b,e]oxepin-2-acetate, cis-11-(2-diethylaminoethyl)imino-6,11-dihydrodibenz[b,e]oxepin-2-acetic acid, trans-11-(2-diethylaminoethyl)imino-6,11-dihydrodibenz[b,e]oxepin-2-acetic acid, methyl cis-11-(3-dimethylaminopropyl)imino-6,11-dihydrodibenz[b,e]oxepin-2-acetate, methyl trans-11-(3-dimethylaminopropyl)imino-6,11-dihydrodibenz[b,e]oxepin-2-acetate, cis-11-(3-dimethylaminopropyl)imino-6,11-dihydrodibenz[b,e]oxepin-2-acetic acid, trans-11-(3-dimethylaminopropyl)imino-6,11-dihydrodibenz[b,e]oxepin-2-acetic acid, methyl cis-3-[11-(2-diethylaminoethyl)imino-6,11-dihydrodibenz[b,e]oxepin-2-yl]-propionate, methyl trans-3-[11-(2-diethylaminoethyl)imino-6,11-dihydrodibenz[b,e]oxepin-2-yl]-propionate, cis-[11-(2-diethylaminoethyl)imino-6,11-dihydrodibenz[b,e]oxepin-2-yl]-propionic acid, trans-[11-(2-diethylaminoethyl)imino-6,11-dihydrodibenz[b,e]oxepin-2-yl]-propionic acid, methyl cis-2-[11-(2-dimethylaminoethyl)imino-6,11-dihydrodibenz[b,e]oxepin-2-yl]-propionate, methyl trans-2-[11-(2-dimethylaminoethyl)imino-6,11-dihydrodibenz[b,e]oxepin-2-yl]-propionate, cis-2-[11-(2-dimethylaminoethyl)imino-6,11-dihydrodibenz[b,e]oxepin-2-yl]-propionic acid, trans-2-[11-(2-dimethylaminoethyl)imino-6,11-dihydrodibenz[b,e]oxepin-2-yl]-propionic acid, methyl cis-11-(2-dimethylaminoethyl)imino-6,11-dihydrodibenz[b,e]oxepin-3-acetate, methyl trans-11-(2-dimethylaminoethyl)imino-6,11-dihydrodibenz[b,e]oxepin-3-acetate, cis-11-(2-dimethylaminoethyl)imino-6,11-dihydrodibenz[b,e]oxepin-3-acetic acid, trans-11-(2-dimethylaminoethyl)imino-6,11-dihydrodibenz[b,e]oxepin-3-acetic acid, methyl cis-11-(3-dimethylaminopropyl)imino-6,11-dihydrodibenz[b,e]oxepin-3-acetate, methyl trans-11-(3-dimethylaminopropyl)imino-6,11-dihydrodibenz[b,e]oxepin-3-acetate, cis-11-(3-dimethylaminopropyl)imino-6,11-dihydrodibenz[b,e]oxepin-3-acetic acid, trans-11-(3-dimethylaminopropyl)imino-6,11-dihydrodibenz[b,e]oxepin-3-acetic acid, methyl 11-(3-dimethylaminopropyl)-6,11-dihydrodibenz[b,e]oxepin-2-carboxylate, 11-(3-dimethylaminopropyl)-6,11-dihydrodibenz-[b,e]oxepin-2-carboxylic acid, 11-(3-dimethylaminopropyl)-6,11-dihydrodibenz-[b,e]oxepin-2-acetic acid, 11-(3-dimethylaminopropylidene)-2-(4,4-dimethyl-2-oxazoline-2-yl)-6,11-dihydrodibenz-[b,e]oxepin, 11-(3-dimethylaminopropyl)-2-(4,4-dimethyl-2-oxazoline-2-yl)-6,11-dihydrodibenz[b,e]oxepin, methyl cis-11-(3-morpholinopropylidene)-6,11-dihydrodibenz[b,e]oxepin-2-carboxylate, methyl trans-11-(3-morpholinopropylidene)-6,11-dihydrodibenz[b,e]oxepin-2-carboxylate, cis-11-(3-morpholinopropylidene)-6,11-dihydro-dibenz[b,e]oxepin-2-carboxylic acid, trans-11-(3-morpholinopropylidene)-6,11-dihydrodibenz[b,e]oxepin-2-carboxylic acid, methyl cis-11-(3-thiomorpholinopropylidene)-6,11-dihydrodibenz[b,e]oxepin-2-carboxylate, methyl trans-11-(3-thiomorpholinopropylidene)-6,11-dihydrodibenz[b,e]oxepin-2-carboxylate, cis-11-(3-thiomorpholinopropylidene)-6,11-dihydrodibenz[b,e]oxepin-2-carboxylic acid, trans-11-(3-thiomorpholinopropylidene)-6,11-dihydrodibenz[b,e]oxepin-2-carboxylic acid, methyl trans-3-[cis-11-(3-dimethylaminopro-pylidene)-6,11-dihydrodibenz[b,e]oxepin-2-yl]-acrylate, methyl trans-3-[trans-11-(3-dimethylaminopro-pylidene)-6,11-dihydrodibenz[b,e]oxepin-2-yl]-acrylate, trans-3-[cis-11-(3-dimethylaminopropylidene)-6,11-dihydrodibenz[b,e]oxepin-2-yl]-acrylic acid, trans-3-[trans-11-(3-dimethylaminopropylidene)-6,11-dihydrodibenz[b,e]oxepin-2-yl]-acrylic acid, methyl cis-11-(3-methylaminopropylidene)-6,11-dihydrodibenz[b,e]oxepin-2-acetate, methyl trans-11-(3-methylaminopropylidene)-6,11-dihydrodibenz[b,e]oxepin-2-acetate, cis-11-(3-methylaminopropylidene)-6,11-dihydrodibenz[b,e]oxepin-2-acetic acid, trans-11-(3-methylaminopropylidene)-6,11-dihydrodibenz[b,e]oxepin-2-acetic acid, methyl cis-11-(3-aminopropylidene)-6,11-dihydrodibenz[b,e]oxepin-2-acetate, methyl trans-11-(3-aminopropylidene)-6,11-dihydrodibenz[b,e]oxepin-2-acetate, cis-11-(3-aminopropylidene)-6,11-dihydrodibenz-[b,e]oxepin-2-acetic acid, methyl trans-11-(3-aminopropylidene)-6,1′-dihydrodibenz[b,e]oxepin-2-acetic acid, or a salt, ester, or amide thereof.

15. The method of claim 11, wherein the mast cell stabilizer is a spleen tyrosine kinase inhibitor.

16. The method of claim 15, wherein the spleen tyrosine kinase inhibitor is 2-[7-(3,4-dimethoxyphenyl)-imidazo[1,2-c]pyrimidin-5-ylamino]-nicotinamide dihydrochloride, 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide, NVP-QAB205, BAY 61-3606, piceatannol, 3,4-dimethyl-10-(3-aminopropyl)-9-acridone oxalate, a purine derivative, or a 1,6-naphthyridine derivative.

17. The method of claim 11, wherein the mast cell stabilizer is a dihydropyridine.

18. The method of claim 17, wherein the dihydropyridine is nicardipine, barnidipine, YC-114, elgodipine, niguldipine or R(−)-niguldipine.

19. The method of claim 11, wherein the mast cell stabilizer is a derivative of alcaftadine.

20. The method of claim 19, wherein the derivative of alcaftadine is a compound of formula (II): 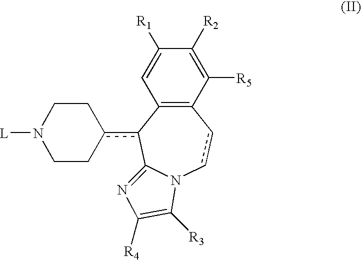
wherein each of the dotted lines independently represents an optional bond; R₁ represents hydrogen, halo, C₁₋₄ alkyl or C₁₋₄ alkyloxy; R₂ represents hydrogen, halo, C₁₋₄ alkyl or C₁₋₄ alkyloxy; R₃ represents hydrogen, C₁₋₄ alkyl, ethenyl substituted with hydroxycarbonyl or C₁₋₄ alkyloxycarbonyl, C₁₋₄ alkyl substituted with hydroxycarbonyl or C₁₋₄ alkyloxycarbonyl, hydroxy C₁₋₄ alkyl, formyl or hydroxycarbonyl; R₄ represents hydrogen, C₁₋₄ alkyl, hydroxy C₁₋₄ alkyl, phenyl or halo; R₅ represents hydrogen, C₁₋₄ alkyl or halo; L represents hydrogen; C₁₋₆ alkyl; C₁₋₆ alkyl substituted with one substituent selected from the group consisting of hydroxy, halo, C₁₋₄ alkyloxy, hydroxycarbonyl, C₁₋₄ alkyloxycarbonyl, C₁₋₄ alkyloxycarbonyl-C₁₋₄ alkyloxy, hydroxycarbonyl C₁₋₄ alkyloxy, C₁₋₄ alkyloxycarbonylamino, C₁₋₄ alkylaminocarbonyl, C₁₋₄ alkylaminocarbonylamino, C₁₋₄ alkylaminothiocarbonylamino, aryl, aryloxy and arylcarbonyl; C₁₋₆ alkyl substituted with both hydroxy and aryloxy; C₃₋₆ alkenyl; C₃₋₆ alkenyl substituted with aryl, or a pharmaceutically acceptable salt or stereochemical isomer thereof.


SEQUENCE LISTING (5 sequences)

SEQ ID NO: 1 - protein, 4 aa, Artificial Sequence
  Description of Artificial Sequence  Synthetic Peptide
YXXL

SEQ ID NO: 2 - DNA, 58 bp, Artificial Sequence
  Description of Artificial Sequence  Synthetic Primer
tgcctgctgcacgaagggaaggtgctgcactatcgcatcgacaaagacagacagggaa

SEQ ID NO: 3 - DNA, 54 bp, Artificial Sequence
  Description of Artificial Sequence  Synthetic Primer
ggaaggtgctgcactatcgcatcgacaaagacaagacagggaagctctccatcc

SEQ ID NO: 4 - DNA, 53 bp, Artificial Sequence
  Description of Artificial Sequence  Synthetic Primer
gcagcgacaaagacaagacagggaagctctccatccccgagggaatataaagc

SEQ ID NO: 5 - DNA, 57 bp, Artificial Sequence
  Description of Artificial Sequence  Synthetic Primer
gggggggctgtcagccatgccgtgtcttgtctttgtcgcttcttgaggagccccccc